(19) United States
(12) Patent Application Publication
(10) Pub. No.: US 2007/0239012 A1
(43) Pub. Date: Oct. 11, 2007

(54) METHOD AND SYSTEM FOR CONTROLLING AN EXAMINATION PROCESS THAT INCLUDES MEDICAL IMAGING

(76) Inventors: Dieter Boeing, Erlangen (DE); Gabriel Haras, Mucke (DE)

Correspondence Address: SCHIFF HARDIN, LLP;PATENT DEPARTMENT, 6600 SEARS TOWER, CHICAGO, IL, 60606-6473, US

(21) Appl. No.: 11/686,680

(22) Filed: Mar. 15, 2007

(30) Foreign Application Priority Data
Mar. 17, 2006 (DE) 10 2006 012 408.1

(51) Int. Cl. A61B 8/00

(52) U.S. Cl. 600/439

(57) ABSTRACT

In a method and system for controlling an examination process that includes medical imaging, which initially a first examination-relevant item of information is input and, based on this first information, first measures of the examination process are automatically established. A first segment of the examination process is subsequently automatically controlled according to at least one part of the first measures. During this first segment or subsequently, a second item of information is input and, based on this second information as well as on the first segment of the examination process, second measures are automatically established. A second segment of the examination process is automatically controlled according to at least one part of these second measures. In the system, an input unit allows input of the information, and a computer automatically establishes the measures, and a control unit controls the examination process.

### BACKGROUND OF THE INVENTION

1. Field of the Invention

The present invention concerns the control of an examination process that encompasses a medical imaging.

2. Description of the Prior Art

Imaging has found a wide proliferation in medicine and is used in diverse ways. It is predominantly used in diagnostics, but also is used as an aid in therapy measures such as, for example, for a biopsy or for catheter navigation. A broad spectrum of imaging apparatuses is used for imaging: ultrasound apparatuses, conventional x-ray projection apparatuses (such as Bucky wall stands or mammography apparatuses), computed tomography systems (CT systems) and magnetic resonance systems (MR systems). The last two cited systems are among the modern 3D systems that are predominantly used in hospitals and larger imaging centers. There are also embodiments of ultrasound apparatuses that can be considered to be among the modern 3D systems.

An imaging device, in particular one of the cited 3D systems, is associated with relatively high purchasing and operating costs. A purchaser (such as, for example, a hospital) therefore has an interest in using the imaging apparatus with optimal efficiency with a high examination throughput in order to recover the costs.

In practice, imaging is a portion of an entire examination process that includes further parts depending on the application field. The examination process connected with the imaging is particularly complicated in the case of modern 3D systems. In practice this leads to a lower examination throughput than would be theoretically possible with the respective imaging apparatus. Moreover, this complex examination process is error prone.

The examination process conventionally is primarily directed by the participating personnel, for example by a radiologist or by a medical-technical radiology assistant. It is in fact thereby possible to design the examination process flexibly; but this procedure requires high level of qualification and a great deal of experience on the part of personnel. Moreover, delays or errors can occur in rarely occurring special cases (which are therefore less likely to be trained for) such as, for example, acute emergency situations.

From WO 2005/051197 a system is known that indicates (dependent in the entered patient data) a selection of examination protocols adapted to the patient data for a diagnosis scanner in order to improve the examination workflow. The patient data include the patient's size and patient's age. Moreover, it is provided to take specifications of the respective radiologist can be taken into account in the selection of the examination protocol. Among other things, it is described that the examination protocol is retrieved from a hospital databank. An exemplary embodiment describes a diagnosis scanner in the form of a CT system; the tube voltage, the tube current being set, for example, set on the respective protocol, and a post-processing is implemented. This system does make the selection of an examination protocol adapted to the patient data and the specifications of the respective radiologist easier; but the determination of an examination protocol makes it more difficult to keep the workflow of the examination process flexible.

### SUMMARY OF THE INVENTION

An object of the present invention is based on the object to enable the control of an examination process of the aforementioned type to proceed in a flexible manner in spite of the control being at least partial automated.

The above object is achieved in accordance with the present invention in a method and system for controlling an examination process that includes medical imaging, wherein examination-relevant item of information is input into a computer and the computer automatically establishes first measures of the examination process that are related to imaging, dependent on the at least one first item of information. A segment of the examination process is then automatically controlled according to at least a portion of the first measures. A second examination-relevant item of information is then entered into the computer after control of the aforementioned segment, and the computer automatically establishes second measures of the examination process that are related to the imaging, dependent on the aforementioned segment and on the second item of information. A further segment of the examination process is then automatically controlled according to at least a portion of the second measures.

By entering examination-relevant information at various points in time into the examination process that embodies a medical imaging in accordance with the invention, a flexible adaptation of the automatic control of the examination processes to the respective additional input information is achieved because in the respective following segment of the examination process the respective preceding segment is taken into account as well, so that it is possible to coordinate the measures of the respective following segment with the measures of the respective preceding segment.

The following describes the case in which at least one first item of information is initially entered and at least one second item of information is entered after the completion of a segment of the examination process, the second item of information being taken into account for a further segment, but the numbering of the at least one item of item of information is selected arbitrarily; i.e., it should not be precluded that further information entered at another point in time of the examination process. A continuous information input is also conceivable. In the same sense the division of the examination process into segments and the division of the measures of the examination process into first or second measures is also arbitrary and merely serves for an illustrative description of the technical explanation of the invention.

Various types of input information are relevant for the examination process, a diagnostic supplication, patient data and/or an examination region are advantageous examples. A medical question arises from this information, to which medical question the respective measures of the examination process are adapted. Partial aspects of a medical question are also adequate. For example, if a suspicion of a stroke exists, it can already be concluded that the examination region should include the head of the patient. In a particularly simple manner the patient data can be at least one result of a preceding examination of the respective patient.

The at least one item of examination-relevant information can be simply input manually, via a measurement or via retrieval from a databank, for example in the form of a hospital information system (HIS) and/or a radiological information system (RIS). The manual input ensues in a user-friendly manner via a selection from at least one predetermined selection list. The selection is particularly simple due to the dependency of the at least one selection list on the respective previously input information; in the event that, for example, “stroke” was previously input as a diagnosis suspicion the selection list for examination regions is adapted such that “leg region” can no longer be selected.

It is possible to provide the input of the second information both as a scheduled part of the examination process and via a user intervention. With regard to a scheduled part of the examination process, the input of the second information can ensue by virtue of the first measure including a requirement for the input of the second information. This makes an interactive control of the examination process possible that, for example, provides branching possibilities that depend on the second information. Input of the second information via a user intervention (action) enables a particularly flexible control that can be adapted on the part of the user to unforeseeable events or results in the course of the examination process. These forms of the input of the second information can be provided alternatively or (advantageously) together.

The second information can be an intermediate result of the first segment that is brought about by the first measure. Naturally in the event of the first measure being only partially implemented, only this respective implemented part of the first measure contributes to the intermediate result. By consideration of the intermediate result, a loss of realizations acquired in the first segment is avoided given an examination process possibly adapted corresponding to the at least one second item of information. Possible duplicate examinations are also avoided in this manner.

Because the second measures are established dependent not only on the first measure and the at least one second item of information but moreover also dependent on the at least one first item of information, it is possible in the control of the examination process to take into account a question that arises from the at least one first item of information and the at least one second item of information.

Beyond the actual imaging, the examination process can include an image representation, an image transfer, and a finding based on the imaging. The association of the apparatuses and workstations relevant for the examination process, as well as the division into work steps necessary in the framework of the examination processes, play a significant role for achieving a fast unimpeded progression (workflow) of the examination process. The measures automatically established according to the inventive method and system therefore advantageously concern at least in part the aforementioned aspects of the examination process beyond the actual imaging.

### DESCRIPTION OF THE DRAWINGS

FIG. 1 is a flowchart of an exemplary embodiment of a previously described method for controlling an examination process in accordance with the invention.

FIG. 2 shows a series of measures of the examination process in accordance with the invention.

FIG. 3 shows, in a perspective view, a CT system with an operating computer suitable for use in accordance with the invention.

FIG. 4 shows in a perspective view, a first space with the CT system of FIG. 3, a second space with a finding computer, and a third space with a PACS in accordance with the invention.

FIG. 5 shows a screen display of a selection menu for selection of information from three lists in accordance with the invention.

FIG. 6 shows a system for controlling the examination process with a communication connection to a patient databank and a user databank, with a connected CT system and with a connection to a network with which moreover the finding computer and the PACS also are connected in accordance with the invention.

### DESCRIPTION OF THE PREFERRED EMBODIMENTS

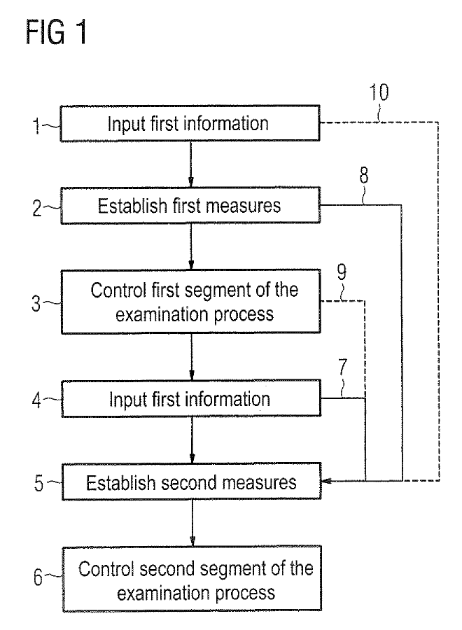

FIG. 1 shows an exemplary embodiment of the inventive method in the sequence of steps 1-6, for which the following abbreviated designations applies:
step 1: input first information,
step 2: establish first measure,
step 3: control first segment of the examination process,
step 4: input second information,
step 2: establish second measure,
step 3: control second segment of the examination process.

It is an object of this method to improve the workflow of the examination process so that the workflow ensues in a time-saving but nevertheless flexible manner.

At least one first, examination-relevant item of information is initially input in step 1, for example a diagnosis suspicion and/or patient data. This input can be made, for example, by a radiologist or an MTRA.

First measures of the examination process that relate to the imaging are automatically established dependent on this first at least one item of information. This establishment is enabled particularly simply by specifications that respectively associate a predetermined configuration (combination) of measures with the at least one item of information; these specifications are stored, for example, in a databank as examination protocols or workflow specifications.

According to at least one part of the previously established first measures, the first segment of the examination process is controlled in step 3; the measures and the correspondingly controlled examination process are explained in detail in relation to subsequent figures.

At least one second examination-relevant item of information is input in step 4. It is on the one hand possible for this input (in particular after only a part of the provided first measure has been implemented in the examination processes) to ensue via an interruption of the examination process by a user, for example the MTRA or the radiologist. Alternatively, the input can be provided as one of the first measures. In the latter cited case, the user can be prompted to make an input by means of a displayed question, for example. This is particularly appropriate when the question is directed toward something that cannot be automatically detected.

Second measures related to the imaging are automatically established in step 5, whereby (beyond the at least one input second item of information) the first segment of the examination process is also taken into account. In this manner it is possible to reasonably adapt the further workflow of the examination process to the preceding workflow. The dependency of the second measure on the at least one second item of information input in step 4 and the first segment of the examination process affected by the measure determined in step 2 is represented in the drawing by the two arrows 7 and 8 respectively from steps 2 and 4 to step 5. Moreover, an intermediate result already gained in the first segment of the examination process is taken into account in step 5 as the second information in order to avoid a duplicate examination for repeated acquisition of results that already exist from the first segment of the examination process. This is indicated with the arrow 9 (shown dashed) from step 3 to step 5. As the arrow 10 from step 1 to step 5 indicates, it is additionally possible to also take at least one first item of information into account in the establishment of the second measure.

The second segment of the examination process is automatically controlled according to at least one part of the previously established second measure. It is possible for an input of at least one examination-relevant item of information also to ensue anew in this further segment, and the previously described method repeats at least in part.

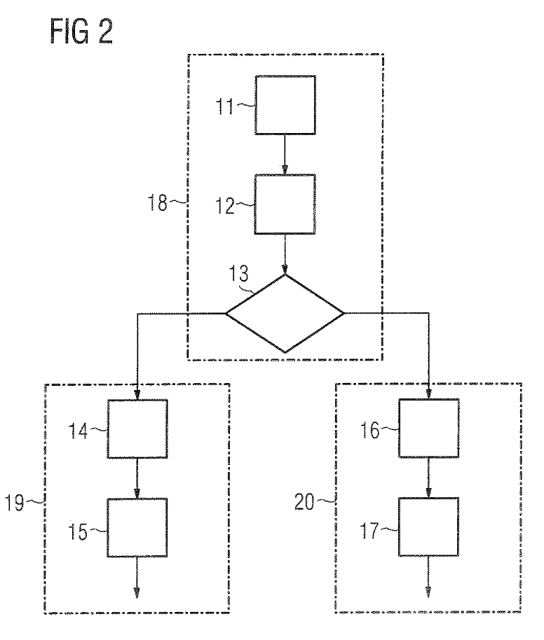

FIG. 2 shows a sequence of measures 11-17 of the examination process that is sub-divided into a first segment 18 and a second segment 19 or 20. The first segment 18 comprises three successive first measures 11-13 that are established based on the input of the at least one first examination-relevant item of information. The first segment 18 begins with two measures 11, 12 (not specified in detail) and ends with a prompt for input of the second examination-relevant item of information as a last measure 13.

Two alternative segments 19 and 20 are provided. Either the measures 14, 15 of the one second segment 19 or the measures 16, 17 of the other second segment 20 are established as second measures, dependent on the first segment 18 and the at least one second item of information. As is indicated by the continuous dashed line from the respective last shown measure 15 or 17, the alternative second segments 19 or 20 can include further measures.

Because the sequence of the measures 11-17 in the segments 18-20 is predetermined in advance by predefined examination protocols, it is possible with particularly little effort to automatically establish the measures 11-17 for the examination process. The measures 11-17 are combined segment-by-segment into an overall sequence. It is comparably advantageous to respectively predetermine in the examination protocols a number of segments 18-20 that (as in FIG. 2) are connected via alternative branches.

The measures 11-17 do not have to be established in a specific temporal sequence but rather can occur simultaneously or without a fixed temporal relation. Moreover, it is naturally also possible that there are more than only two alternatives for the second segment 19 and 20.

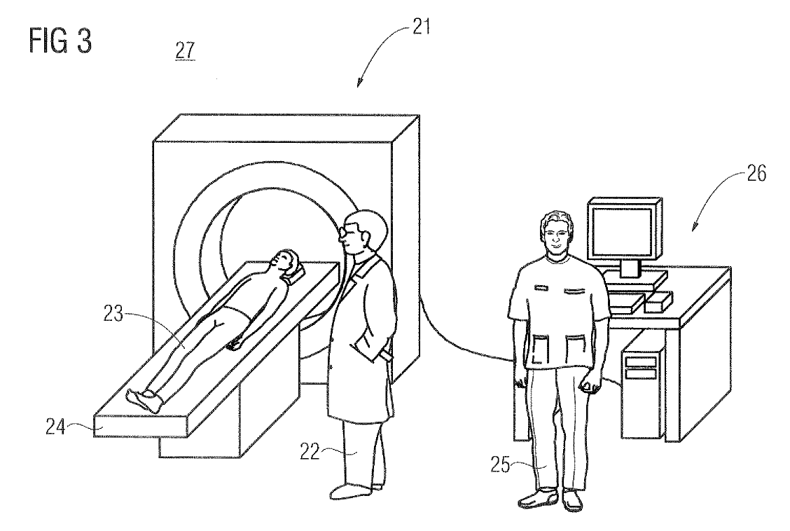

Which measures 11-17 can be provided is explained using FIGS. 3-5 in the framework of practice-related examples.

FIG. 3 shows a CT system 21 in a typical usage environment. An MTRA 22 attends to a patient 23 who is located on a bed 24 of the CT system 21. A radiologist 25 operates the CT system 21 via the operating computer 26.

An input of the at least one item of information is particularly time-saving at least in part due to a retrieval of at least one user preference. This user preference is, for example, centrally stored; it can either be derived from prior input by the respective user 22 or 25 in comparable situations or be manually expanded and processed by the respective user. In the event that a user 22 or 25 predominantly makes the same selection of apparatus parameters at a specific point of the examination process, these apparatus parameters are then automatically selected in the subsequent examinations. The method is thus adaptive (capable of learning) in the manner of a neural network and allows the number of routinely identical inputs to be reduced; for example, the method can be controlled with the aid of an integrated expert system.

At the beginning of the examination the patient 23 is registered at the CT system 21; this registration is automatically controlled as one of the measures 11-17. For this purpose, patient data that allow an identification of the patient 23 are input, for example at the operating computer 26. In the event that no patient data about this patient 23 are present in a (possibly central) patient databank, it is provided to complete the data set of the patient 23 in the patient databank. The patient databank can be executed, for example, as a hospital information system (HIS) or as a radiological information system (RIS). For example, laboratory values and results of preceding examinations are also stored as patient data; these patient data can be retrieved at the operating computer 26. For example, it is possible to establish that the patient 23 has a reduced kidney function, such that no kidney-damaging contrast agent should be administered. The information about a reduced kidney function can also be taken into account without user intervention in the establishment of the measures.

A further measure provides to automatically acquire patient data via a measurement and to input said patient data as information. For example, the blood pressure, the heart rate and/or the body size can be automatically acquired.

An input of information in the form of a desired examination region is provided as a further measure. This input ensues, for example, on the basis of an input request that is displayed in the framework of the measure for the radiologist 26. Such an input can be omitted when the appropriate examination region is clear from other information. In such a case it is possible to also take the desired examination region into account in the establishment of the measure without further input.

The measures 11-17 include a setting of imaging parameters so that an error-prone manual setting of these imaging parameters is omitted. The image acquisition can be controlled in a low-effort manner via a measure in the form of a selection of an image acquisition protocol; the image acquisition protocol can also comprise specifications for the imaging parameters.

Moreover, the measures 11-17 includes a control of a dose automatic which adapts the dose of x-ray radiation employed for imaging to the respective patient 23. This adaptation ensues based on the body size of the patient and the weight of the patient 23 or (more precisely) based on a topogram of the respective patient 23 generated with the CT system 21. The topogram is an overview image of the respective patient 23 that is generated with a comparably low dose. This overview image enables a setting of the x-ray dose and a limitation of the examination region. The dose automatic can particularly precisely set for a particular x-ray dose based on the topogram. The generation of the topogram is advantageously also the subject matter of a further measure. The dose automatic prevents the patient 23 from being exposed to too high a radiation exposure that is too high.

The measures 11-17 also include an acquisition of image data of a provided examination region of the respective patient 23. In practice the case occasionally occurs that image data should be acquired not only from one but rather from two or more examination regions. When two partial examination regions intersect, to avoid a duplicate acquisition of the intersection region it is provided to acquire a total examination region comprising the two partial examination regions. An acquisition of image data of an examination region respectively comprising a partial examination region input as an examination-relevant first or second item of information is therefore provided as a possible measure.

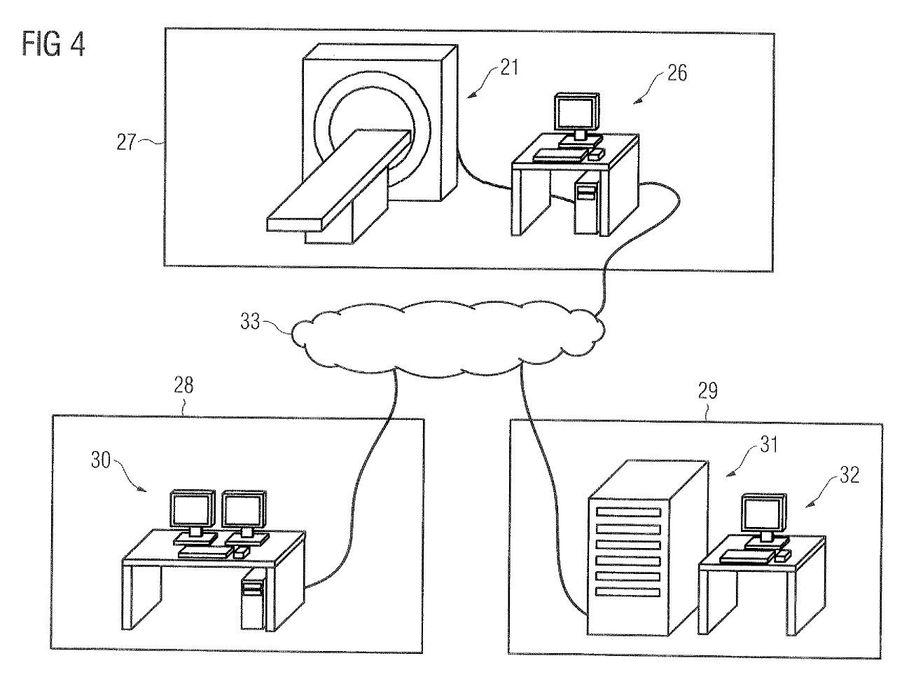

In a perspective view FIG. 4 shows three spaces 27-29. The CT system 21 shown in FIG. 3 with the operating computer 26 associated with this is located in the first space 27. Since the examination of the patient is conducted in the first space 27, the first space 27 is also called an examination station in the following. The second space 28 is a finding system with a finding computer 30. The third space comprises a PACS 31 (“picture archiving and communication system”) on which image data are stored for a later access; for maintenance purposes, the PACS 31 is connected with a service computer 32 via a communication connection. The operating computer 26, the finding computer 30 and the PACS 31 can exchange data over a network 33. The arrangement of the three spaces 27-29 and the network 33 shown in FIG. 4 is part of a hospital in which further imaging systems and further finding stations are present.

The image data acquired with the CT system 21 are transferred over the network 33 to the finding computer 30; a manual initiation of the transfer is done away with via a measure directed towards the transfer. The automatic selection of the respective finding computer 30 depends on the respective radiologist 25 conducting the examination and the respective availability of the finding computer 30. An identification of finding computer 30 and/or in which finding station 28 the image data have been transferred is shown to the radiologist 25 at the operating computer 26 in the examination space 27.

The measures 11-17 include a generation of at least one image representation based on the image data as well as a processing of the image representation. It is thus possible, for the radiologist 25 to make diagnosis-relevant conclusions using the image representation without having to manually start the generation of the image representation and/or its processing beforehand. Since the generation and processing of the image representation are often very time-consuming, a wait time is omitted in this manner; when the radiologist 25 reaches the finding station 28, he can immediately assess the corresponding prepared image representation of the examination region. The type of the generation and the processing of the image representation is dependent on the preferences (possibly stored in a user databank) of the radiologist 25 and on the respective input at least one item of information. Depending on which clinical question arises from this at least one item of information, the image representation is adapted. The correlation between the at least one input item of information and the respective generation and/or processing of the image representation can also be stored in the examination protocols. Among other things, what is known as a “post-processing”, an adaptation of the viewing angle, what is known as a “computer-aided diagnosis” (CAD for short), a segmentation of the image representation as well as a removal of bones from the image representation (also called “bone removal”) belong to the possible processing measures.

Naturally it is also possible for the image representation to be displayed on the operating computer 26; the radiologist 25 or the MTRA 22 is then able to assess whether the acquisition of the image data has succeeded in a desired manner.

According to a further measure, a transfer of the image data to the PACS 31 is provided; such an automatic transfer ensures a reliable saving of the image data for a later access.

The measure 11-17 also advantageously include an (in particular automatic) generation of a finding template. In this exemplary embodiment this finding template is displayed with the image representation on the finding computer 30 and can be immediately filled out by the radiologist 25. The finding template is generated particularly simply by accessing a collection of finding templates. The selection of the respective finding template conforms to the preferences of the radiologist 25 and/or to the clinical question that results from the at least one input item of information. This selection can also be predetermined in the examination protocol. The collected finding templates can be both generic templates stored in advance and templates generated from preceding findings.

The generation of the finding based on the finding template is significantly simplified via a measure for adoption into the finding template of a result of the examination process that is procured via the respective preceding measures 11-17. Moreover, pointers (references) to the implemented examination steps and the preceding measures 11-17 can also be automatically incorporated into the finding template. The result can exist, for example, in the form of a measurement result. As a further completion aid it is possible to offer the radiologist 25 text blocks with standard formulations for the respective clinical question for selection.

Further measures 11-17 concern the space association and apparatus association. By an automatic association of at least one apparatus provided for implementation of the examination process, on the one hand an apparatus appropriate for the examination is selected dependent on the at least one item of information, and the apparatus utilization is increased due to a better coordination with examinations ensuing in parallel. The latter is achieved particularly simply by a central control of the examinations ensuing in parallel. For example, the finding computer 30 or the CT system 21 is to be understood as an apparatus here. An automatic association of at least one workstation provided for implementation of the examination process is advantageous in a similar manner. It is thus possible to more efficiently use the existing workstations and to avoid a double booking of a workstation (in particular given a central control of the examination process). The spaces 27-29 are examples of workstations, but a region within a space or a writing table can also be considered as a workstation. The associated workstation or the associated apparatus is indicated on the operating computer 26 or on the finding computer 30. Moreover, a display on a mobile apparatus that is carried by the radiologist 25 or the MTRA 22 is also conceivable.

With further measures 11-17 that have an influence on the personnel work control, it is possible to associate concomitant work tasks with the examination process in a manner that is time-saving and reliable. At least one work step related to the imaging (such as, for example, a pointer to the positioning of the patient 23 on the bed 24 of the CT system 21) is indicated.

A further example of a work step indicated in the framework of the aforementioned measure is an administration of an imaging-relevant contrast agent to the patient 23. It is thereby possible to also display the respective quantity of the contrast agent adapted to the patient 23. In a similar manner it is advantageously provided to indicate a work step for treatment of the patient 23; the treatment can, for example, ensue in the form of an administration of a medicine with adapted dose.

A particularly efficient division of labor is possible by a display of a pointer to a person 22 or 25 provided for implementation of the respective at least one work step. This division can be predefined in the examination protocols, for example. An automation of this work division can in particular be advantageously applied in the complex work processes of a hospital. For example, specific work steps are explicitly associated with the radiologist 25 or the MTRA 22.

Further work steps concern the manual image post-processing and the preparation of a finding. For example, the MTRA 22 can be instructed to uncover a specific organ in the three-dimensional image representation for better visibility for the radiologist 25 and to make specific entries in the finding. In this manner it is possible to unburden the radiologist of tasks that can also be conducted by assisting personnel.

A further work step is a provided follow-up examination; via a pointer to the follow-up examination it is possible for the user 22 or 25 to make an appointment with the patient 23. Such a pointer can, for example, be indicated at the operating computer 26 arranged in proximity to the CT system 21 while the patient 23 remains in proximity to the CT system 21 for the examination. Moreover, a reservation of the necessary personnel and the necessary apparatus for the follow-up examination an immediate planning of a follow-up examination is possible via a connection to a planning system (for example in the form of the HIS).

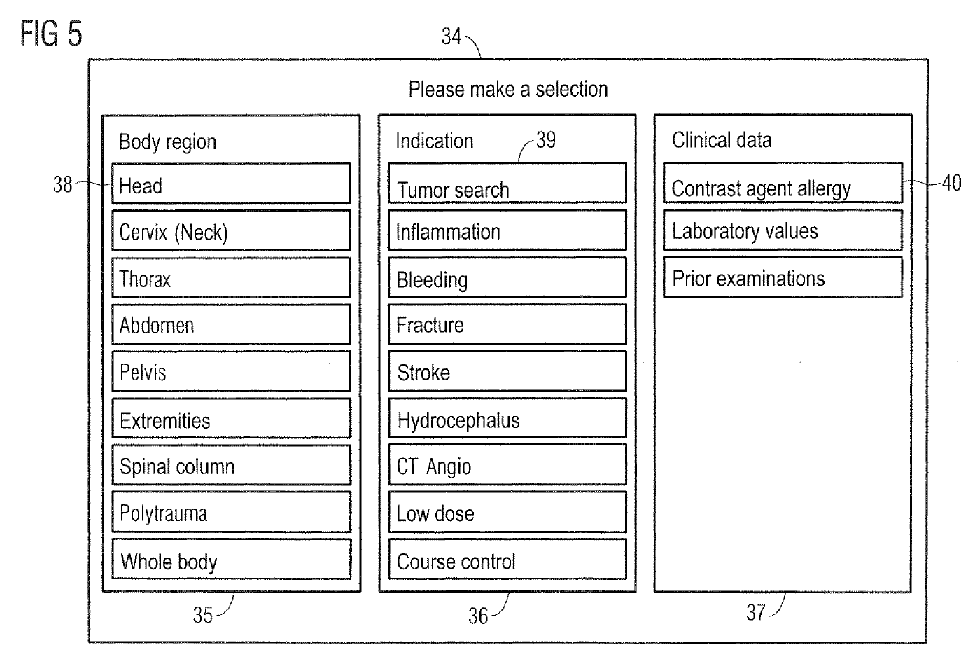

FIG. 5 shows a screen display 34 of a selection menu for selection of information that can be selected from three selection lists 35-37. The body region can be selected from the first selection list 35, the indication from the second selection list 36 and clinical data from the third selection list 37.

The first selection list 35 comprises the following entries 38:
head,
cervix or neck,
thorax or chest,
abdomen,
pelvis,
extremities,
spinal column,
polytrauma,
whole body.

The second selection list 36 includes the following entries 39:
tumor search,
inflammation,
bleeding,
fracture,
stroke,
hydrocephalus,
CT angio,
low dose,
course monitoring.

The third selection list 37 includes the following entries 40:
contrast agent allergy,
laboratory values,
prior examinations.

The three selection lists 35-37 are clearly arranged next to one another and are overwritten with the request “Please make a selection”.

The entry “low dose” specifically concerns an imaging with an x-ray apparatus or, respectively, a CT system 21. The entry “CT angio” is specific to an imaging with a CT system 21.

It is possible to select more than one entry 38-40 or no entry 38-40 at all from a selection list 35-37. A particularly simple selection is enabled in that the predetermined selection lists 35-37 are adapted (in terms of their selection possibilities) dependent on the respective previously input information. For example, in the event that initially the entry “tumor search” is selected, the possibility to select the entry “low dose” is then omitted since an acquisition of image data with a high resolution and therefore with a comparably high x-ray dose must ensue given a tumor search. For example, the entry “low dose” can moreover be faded out or be altered in its representation so that the user (for example in the form of the radiologist 25) can recognize that this entry can no longer be selected.

The selection of some of the entries 38-40 has as a consequence the selection or a acceleration of a selection of at least one further entry. In the event of a selection of the entry “stroke” in the selection list “indication”, the entry “head” is automatically also selected as well in the selection list “body region” since a stroke can only occur in the head. The selection is further simplified for the respective user 22 or 25 via the previously-described dependency in the selection of the entries 38-40.

Moreover, it is advantageously provided to adapt (in terms of its selection possibility) the at least one predetermined selection list dependent on the at least one user preference. Entries that the user 22 or 25 typically does not select are either not displayed at all or are presented as not selectable, in contrast to the remaining entries. The displayed selection menu is clear in this manner and allows a fast selection of the entries 38-40. Moreover, one or more entries can be preselected dependent on the at least one user preference. The user 22 or 25 can accept this pre-selection (for example with an acknowledgement entry (not shown)) or select entries deviating from the pre-selection.

In the following a practice-related example of an examination process is described.

A patient 23 is delivered to a hospital with a diagnosis suspicion of a cerebral hemorrhage. The patent 23 is registered, whereby the patient data (such as, for example, name, birth date and patient identification) are input insofar as these patient data are not already stored in the HIS of the hospital. As first information these patient data as well as further patient data stored in the HIS form the basis of the subsequent method.

The radiologist 25 examines the patient 23 and affirms the diagnosis suspicion. With the aid of the screen display 34 shown in FIG. 5, he or she thereupon inputs further first information in that he selects the body region “head” as well as the indication “bleeding”. In a further screen display (not shown) the radiologist 25 selects the precise head region “brain”. Based on this first input by the radiologist, a specific examination protocol that establishes first measures of the examination process is automatically selected from a plurality of examination protocols stored in advance. The examination protocol is automatically selected such that it best approximates the respective question. A first segment of the subsequent examination process is controlled dependent on the measures established in this case.

The examination protocol automatically associates an examination station 27, a radiologist 25 as well as an MTRA 22 with the examination. This association conforms with the respective qualification of the personnel required for this examination, the respective equipment of the examination station necessary for this examination and the availability of the personnel and the accommodations. The association is displayed to the personnel.

The actual imaging of the examination ensues in the examination station 27 with the CT system 21 shown in FIG. 3. Work steps for preparation of the patient 23 are displayed to the MTRA 22; among other things, these work steps comprise a suitable positioning of the patient 23 on the bed 24 of the CT system 21.

A duration for the acquisition of a topogram is automatically set dependent on the stored patient data, in particular dependent on the size of the patient 23. The automatic acquisition of the topogram ensues according to this measure, which topogram is stored in a local storage of the operating computer 26 and/or in a central storage of the PACS 31. According to further measures of the examination protocol, based on the topogram the examination region of the patient 23 is limited to the brain of the patient 23, and a dose automatic is activated that adapts the x-ray dose to the respective patient 23.

Image data of the examination region are acquired according to a further measure. These image data are stored in the local storage in the operating computer 26; a further measure provides to reconstruct an image representation of the examination region starting from the image data, which image representation is stored both in the local storage of the operating computer 26 and in the central storage of the PACS 31. The reconstruction ensues with reconstruction parameters adapted to the diagnosis suspicion “cerebral hemorrhage”.

For quality control and for a first intermediate diagnosis, the image representation is displayed at the operating computer 26 corresponding to a further measure. A further one of the first measures allows the radiologist 25 to input the second information in the form of a quality assessment. If the radiologist 25 inputs that the quality is not sufficient, the acquisition of the image data is then repeated with modified parameters. Further second information for improved limitation of the examination region or for adaptation of the resolution of the image data can thereby be manually input.

If the radiologist establishes (for example using the displayed image representation) that no cerebral hemorrhage actually exists, but rather that a stroke exists in the form of an ischemia, he or she can interrupt the examination process in order to input second information in the form of a changed indication. The radiologist 25 thereby calls up the menu shown in FIG. 5 in order to change the indication “bleeding” to “stroke”. A different examination protocol is automatically called up based on this; since image data of the same examination region were already acquired in the framework of the first measures, a new acquisition of image data is omitted in the second measures established according to the new examination protocol. The already acquired image data are reconstructed according to one of the second measures with reconstruction parameters adapted to the diagnosis suspicion “stroke”, whereby, for example, the slice thickness is reduced relative to the preceding reconstruction. Instead of a retrieval of a further examination protocol a branching can also ensue within the same examination protocol; this has no influence on the actual sequence of the measures (11-17), but rather merely has an effect on the manner of the administration of the examination protocols.

A further second measure is to display to the MTRA 22 a work step for administration of a contrast agent to the patient 23. According to a next second measure, image data of the patient 23 with the contrast agent are acquired. A subtraction image representation according to the method of digital subtraction angiography (DSA) is reconstructed based on the image data of the patient 23 without contrast agent and the image data of the patient 23 with contrast agent. The image representation acquired via this measure is stored both in the operating computer 26 and in the PACS 31.

The subtraction image representation is transferred in further second measures to the finding station 28, displayed on the finding computer 30 there and assessed by the radiologist 25 who receives a direction to the corresponding finding station 28. According to a further measure a finding template that is processed by the radiologist 25 is automatically generated at the finding computer 30. A vessel occlusion occurring given an ischemia is well recognizable from the subtraction image representation for the radiologist 25. The radiologist 25 enters into the finding this diagnosis gained in the consideration of the image representation. Further treatment suggestions are possibly offered to the radiologist 25 according to a further measure based on the diagnosis and the preceding measures.

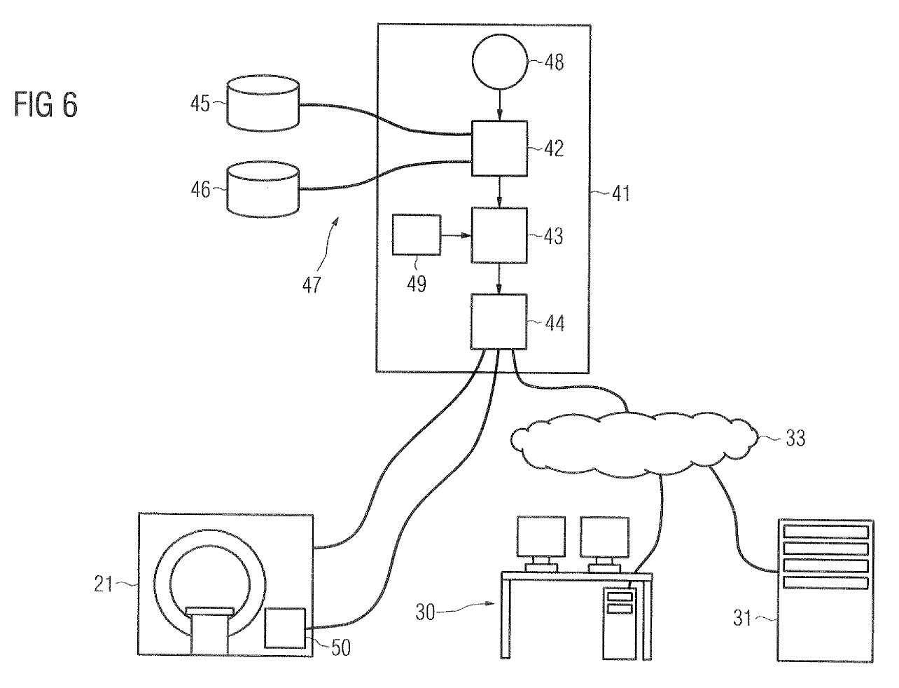

FIG. 6 shows a system 41 for controlling the examination process. This system 41 comprises an input unit 42 for input of at least the examination-relevant information, a computer 43 for automatic determination of the measures 11-17 of the examination process that are related to the imaging, which determination is dependent on the respective input at least one item of information, and a control unit 44 for automatic control of the respective segment 18-20 of the examination process according to at least one part of the respective established measures 11-17. The system 41 is set up such that it is suitable for implementation of the method described above.

The system 41 can include both hardware components and software components For example, it is possible for the system 41 to be a component of the operating computer 26 on which a computer program product is installed which executes the method described previously. This computer program product can alternatively be installed on a central computer in the hospital.

Both the at least one first item of information and (after an interim control of one of the segments 18-20 of the examination process) the at least one second item of information can be input with the aid of the input unit 42. Depending on the at least one first item of information, the computer 43 initially establishes first measures 11-13 of the examination process that are related to the imaging. The computer 43 establishes second measures 14-17 of the examination process dependent on these first measures 11-13 and on the at least one second item of information.

Both a patient databank 45 and a user databank 46 are connected with the input unit 45 via a communication connection 47. Via a data retrieval from the patient databank 45 and from the user databank 46, the patient data on the one hand or, respectively, the user preferences on the other hand can respectively be input as examination-relevant information. Both a local storage of the databanks 45 and 46 and a central storage (for example in the central computer in the hospital) are possible. The central storage enabled via the communication connection 47 makes the use of the databanks 45 and 46 at various examination stations 27 easier. The administration of the examination protocols can also ensue centrally in a manner comparable with the databanks 45 and 46.

In this exemplary embodiment the input unit 42 possesses a measurement sensor 48 such that the input unit 42 is fashioned in the form of a measurement unit with which the at least one examination-relevant item of information can be input via an (in particular automatic) measurement. The measurement sensor 42, 48 can, for example, be fashioned for measurement of a blood pressure of the patient 23.

The input unit 42 is additionally or alternatively designed for a manual input of the at least on item of information. For example, the input unit 42 is fashioned in the form of a keyboard; this enables for the respective user 22 or 25 a flexible input of a broad spectrum of information.

A display unit 49 of the system 41 enables a particularly user-friendly interaction with the system 41. For example, a display of the at least one selection list 35-37 is possible at the display unit 49; the selection of at least one item of information is then possible in a particularly simple manner using the input unit 42, in the form of a selection of respectively at least one entry 38-40 of the selection list 35-37. This works particularly simply with a computer mouse as a part of the input unit 42. The display unit 49 is fashioned in a simple manner as a monitor of the operating computer 26. A display at the system 41 of the at least one work step and moreover of the person 22 or 25 provided for implementation of the respective work step is possible with the aid of the display unit 49.

The CT system 21 that is controlled by the control unit 44 of the system 41 is connected to the system 41; in this manner, for example, it is possible to acquire image data of the limited examination region with the CT system 21 in the sense of a previously-established measure. Moreover, the system 41 is connected with the dose automatic 50 of the CT system, which dose automatic 50 is controlled by the control unit 44.

Instead of the CT system 21 it is naturally also conceivable to additionally or alternatively connect further imaging apparatuses with the system 41. A standard interface (not shown) can be provided at the system 41 for this.

Via the network 33 the system 41 is in the position to exchange data with the finding computer 30 and the PACS 31; it is by possible for the system 41 to, 31, send image data acquired with the CT system 21 to the finding computer 30 and/or to the PACS 31.

In summary, in accordance with the invention initially a first examination-relevant item of information is input and, based on this first information, first measures of the examination process are automatically established; and a first segment of the examination process is subsequently automatically controlled according to at least one part of the first measures. During this time or subsequently, a second item of information is input and, based on this second information as well as on the first segment of the examination process, second measures are automatically established; and a second segment of the examination process is automatically controlled according to at least one part of these second measures. Moreover, at least one further embodiment of the invention concerns a system with an input unit for input of information, with a computer for automatic establishment of measures, and with a control unit for controlling the examination process, with the cited units of the system interacting according to the basic description in relation to the method. It is thereby possible to control an examination process in a flexible manner in spite of an at least partial automation.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for controlling an examination process that includes medical imaging, comprising the steps of:
entering at least one first examination-relevant item of information into a computer;
in said computer, automatically establishing first measures of said examination process related to said medical imaging, dependent on said at least one first examination-relevant item of information;
automatically controlling a segment of the examination process according to at least a portion of said first measures;
after controlling said segment of the examination process, entering at least one second examination-relevant item of information into said computer;
in said computer, automatically establishing second measures of said examination process related to said medical imaging, dependent on said segment of the examination process and on said at least one second examination-relevant item of information; and
automatically controlling a further segment of the examination process according to at least a portion of said second measures.

2. A method as claimed in claim 1 comprising selecting either of said first examination-relevant item of information or said second examination-relevant item of information from the group consisting of a diagnosis suspicion, patient data, a result of a preceding examination of a patient, and an examination region of a patient.

3. A method as claimed in claim 1 comprising entering either of said first examination-relevant item of information or said second examination-relevant item of information into said computer by a procedure selected from the group consisting of electronic retrieval from a patient data bank, a manual measurement conducted on a patient, an automatic measurement conducted on a patient, electronic retrieval of a user preference, manual input, and manual selection from a predetermined selection list.

4. A method as claimed in claim 1 comprising entering at least one of said first examination-relevant item of information or said second examination-relevant item of information into said computer by a selection from an adaptable selection list that is adaptable according to an adaptation criterion selected from the group consisting of previously entered information and at least one user preference.

5. A method as claimed in claim 1 comprising including, in said first measures, a request for input of said second examination-relevant item of information.

6. A method as claimed in claim 1 comprising manually entering said second examination-relevant item of information.

7. A method as claimed in claim 1 comprising entering, as said second examination-relevant item of information, an intermediate result of said segment of said examination process produced by at least a portion of said first measures.

8. A method as claimed in claim 1 comprising establishing said second measures in said computer additionally on said first information-relevant item of information.

9. A method as claimed in claim 1 comprising establishing, as said second measures, measures selected from the group consisting of setting of imaging parameters of an imaging apparatus, selecting an image acquisition protocol of an imaging apparatus, activating a dose automatic of an X-ray apparatus for said imaging, activation of a dose automatic of a computed tomography apparatus for said imaging, acquisition of a topogram, and acquisition of image data of an examination region of a patient.

10. A method as claimed in claim 1 comprising entering as said first examination-relevant item of information, a first portion of an examination region to be included in said medical imaging, and entering, as said second examination-relevant item of information, a second portion of an examination region to be included in said medical imaging, and wherein said second measures comprise acquiring image data from an examination region comprising said first partial examination region and said second partial examination region.

11. A method as claimed in claim 10 comprising establishing, as said second measures, measures selected from the group consisting of generating at least one image from said image data, processing an image generated from said image data, transferring said image data to another computing device, and transferring said image data to a data storage unit.

12. A method as claimed in claim 1 comprising establishing, as said second measures, measures selected from the group consisting of independently generating a finding template, generating a finding template by access to a stored collection of finding templates, and identifying a result of said examination process and incorporating said result into a finding template.

13. A method as claimed in claim 1 comprising establishing, as said second measures, measures selected from the group consisting of identifying at least one apparatus for participating in said examination process, and identifying at least one computing workstation for participation in implementation of said examination process.

14. A method as claimed in claim 1 comprising establishing, as said second measures, identifying at least one imaging apparatus for conducting said medical imaging in said examination process.

15. A method as claimed in claim 1 comprising establishing, as said second measures, displaying at least one work step relating to said medical imaging.

16. A method as claimed in claim 15 comprising selecting said work step from the group consisting of designating a person to implement said work step, administering an imaging-relevant contrast agent to a patient, manual image post-processing, administering a medicine to a patient, preparation of a finding resulting from said examination process, and recommending a follow-up exam.

17. A method as claimed in claim 1 comprising, in said computer, establishing at least one of said first measures or said second measures by automatically selecting a template comprising a plurality of available measures.

18. A system for controlling an examination process that includes medical imaging, comprising:
a computer;
an input unit connected to the computer allowing entry of at least one first examination-relevant item of information into said computer;
said computer automatically establishing first measures of said examination process related to said medical imaging, dependent on said at least one first examination-relevant item of information;
a control unit in communication with said computer that automatically controls a segment of the examination process according to at least a portion of said first measures;
said input unit, after said control unit controls said segment of the examination process, allowing entry of at least one second examination-relevant item of information into said computer;
said computer automatically establishing second measures of said examination process related to said medical imaging, dependent on said segment of the examination process and on said at least one second examination-relevant item of information; and
said control unit automatically controlling a further segment of the examination process according to at least a portion of said second measures.

19. A system as claimed in claim 18 wherein said input unit allows selection of either of said first examination-relevant item of information or said second examination-relevant item of information from the group consisting of a diagnosis suspicion, patient data, a result of a preceding examination of a patient, and an examination region of a patient.

20. A system as claimed in claim 18 wherein said input unit allows entry of either of said first examination-relevant item of information or said second examination-relevant item of information into said computer by a procedure selected from the group consisting of electronic retrieval from a patient data bank, a manual measurement conducted on a patient, an automatic measurement conducted on a patient, electronic retrieval of a user preference, manual input, and manual selection from a predetermined selection list.

21. A system as claimed in claim 18 wherein said input unit allows entry of at least one of said first examination-relevant item of information or said second examination-relevant item of information into said computer by a selection from an adaptable selection list that is adaptable according to an adaptation criterion selected from the group consisting of previously entered information and at least one user preference.

22. A system as claimed in claim 18 wherein said computer includes, in said first measures, a request for input of said second examination-relevant item of information.

23. A system as claimed in claim 18 wherein said input unit allows manually entry of said second examination-relevant item of information.

24. A system as claimed in claim 18 wherein said input unit allows entry, as said second examination-relevant item of information, of an intermediate result of said segment of said examination process produced by at least a portion of said first measures.

25. A system as claimed in claim 18 wherein said computer establishes said second measures in said computer additionally on said first information-relevant item of information.

26. A system as claimed in claim 18 wherein said computer establishes, as said second measures, measures selected from the group consisting of setting of imaging parameters of an imaging apparatus, selecting an image acquisition protocol of an imaging apparatus, activating a dose automatic of an X-ray apparatus for said imaging, activation of a dose automatic of a computed tomography apparatus for said imaging, acquisition of a topogram, and acquisition of image data of an examination region of a patient.

27. A system as claimed in claim 18 wherein said input unit allows entry, as said first examination-relevant item of information, of a first portion of an examination region to be included in said medical imaging, and entry, as said second examination-relevant item of information, of a second portion of an examination region to be included in said medical imaging, and wherein said second measures comprise acquiring image data from an examination region comprising said first partial examination region and said second partial examination region.

28. A system as claimed in claim 27 wherein said computer establishes, as said second measures, measures selected from the group consisting of generating at least one image from said image data, processing an image generated from said image data, transferring said image data to another computing device, and transferring said image data to a data storage unit.

29. A system as claimed in claim 18 wherein said computer establishes, as said second measures, measures selected from the group consisting of independently generating a finding template, generating a finding template by access to a stored collection of finding templates, and identifying a result of said examination process and incorporating said result into a finding template.

30. A system as claimed in claim 18 wherein said computer establishes, as said second measures, measures selected from the group consisting of identifying at least one apparatus for participating in said examination process, and identifying at least one computing workstation for participation in implementation of said examination process.

31. A system as claimed in claim 18 wherein said computer establishes as said second measures, identifying at least one imaging apparatus for conducting said medical imaging in said examination process.

32. A system as claimed in claim 18 wherein said computer establishes, as said second measures, displaying at least one work step relating to said medical imaging.

33. A system as claimed in claim 32 wherein said computer selects said work step from the group consisting of designating a person to implement said work step, administering an imaging-relevant contrast agent to a patient, manual image post-processing, administering a medicine to a patient, preparation of a finding resulting from said examination process, and recommending a follow-up exam.

34. A system as claimed in claim 18 wherein said computer, establishes at least one of said first measures or said second measures by automatically selecting a template comprising a plurality of available measures.
